(19) United States
(12) Patent Application Publication
(10) Pub. No.: US 2024/0307001 A1
(43) Pub. Date: Sep. 19, 2024

(54) SYSTEM AND METHOD FOR TRAINING A MODEL TO MONITOR HEALTH PARAMETERS

(71) Applicant: KNOW LABS, INC., Seattle, WA (US)

(72) Inventor: JOHN CRONIN, Seattle, WA (US)

(21) Appl. No.: 18/601,481

(22) Filed: Mar. 11, 2024

Related U.S. Application Data
(60) Provisional application No. 63/490,654, filed on Mar. 16, 2023.

(51) Int. Cl. A61B 5/00; A61B 5/021; A61B 5/05

(52) U.S. Cl. CPC A61B 5/7267; A61B 5/021; A61B 5/05

(57) ABSTRACT

A method for training a model to monitor the health parameters of a user. The method includes monitoring a blood pressure of a user using a control blood pressure monitoring system; receiving control data corresponding to the monitoring using the control blood pressure monitoring system; inputting a frequency range and a frequency-time event and calculating a randomized frequency range; incrementing the frequency-time event; transmitting the calculated randomized frequency and matching the frequency-time limit with the stored frequency-time limit; receiving the calculated randomized frequency range corresponding to radio waves that have reflected from blood in a blood vessel of the user; generating training data by combining the control data with the randomized frequency range in a time synchronous manner; and training a model using the training data. The trained model correlates frequency range to values indicative of the user's blood pressure.

### FIELD

The present disclosure is generally related to systems and methods of monitoring health parameters and, more particularly, relates to a system and a method of monitoring real-time blood pressure levels using radio frequency signals.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

High blood pressure, also known as hypertension, can lead to several health problems. When blood pressure is consistently elevated, it can put extra strain on the heart and blood vessels. Over time, this can increase the risk of several serious and potentially life-threatening conditions, including:

Heart disease: High blood pressure can increase the risk of heart attack, heart failure, and stroke.

Kidney damage: The kidneys filter waste from the blood, and high blood pressure can damage the blood vessels in the kidneys, leading to kidney disease.

Eye damage: High blood pressure can lead to vision loss and even blindness by damaging the blood vessels in the eyes.

Cognitive decline: Some studies have suggested that high blood pressure may be associated with a decline in cognitive function and an increased risk of dementia.

Sexual dysfunction: High blood pressure can also lead to sexual problems in both men and women.

Therefore, it is important to maintain a healthy blood pressure level to reduce the risk of these and other health problems.

Analytes are substances in the blood that can be measured to assess various aspects of health. In the context of blood pressure, some of the most commonly measured analytes include:

Sodium: Sodium is a mineral that helps regulate blood pressure. High levels of sodium in the blood can increase blood pressure, while low levels can lead to decreased blood pressure.

Potassium: Potassium is another mineral that helps regulate blood pressure. It works in conjunction with sodium to maintain healthy blood pressure levels.

Cholesterol: High levels of cholesterol in the blood can increase the risk of heart disease and stroke, which are both related to high blood pressure.

Triglycerides: Triglycerides are a type of fat in the blood that can contribute to heart disease and stroke if they are present at high levels.

C-reactive protein (CRP): CRP is a protein produced by the liver in response to inflammation. High levels of CRP have been linked to an increased risk of heart disease and stroke, which are both related to high blood pressure.

Homocysteine: Homocysteine is an amino acid in the blood that has been linked to an increased risk of heart disease and stroke.

Renin: Renin is an enzyme produced by the kidneys that helps regulate blood pressure. Abnormal levels of renin in the blood can indicate problems with blood pressure regulation.

Aldosterone: Aldosterone is a hormone produced by the adrenal glands that helps regulate blood pressure by controlling the balance of sodium and potassium in the body.

These are some of the analytes that can be related to blood pressure, and their levels can provide important information about the health of the cardiovascular system.

Additionally, some non-invasive devices are used to determine the health parameters of a user using radio waves. These devices involve transmitting radio waves into the user and receiving reflected wavelengths to determine the health parameters. Also, some received wavelengths are filtered using beamforming and the Doppler effect, which are not required to determine blood pressure levels. Moreover, filtering out unnecessary reflected radio waves does not enhance the accuracy of monitoring the blood pressure level. A training data set is also employed to further aid in enhancing the accuracy of determining the accurate blood pressure level. A stepped frequency transmission approach is used in the prior art to generate the training data set. In the stepped frequency approach, a sensor system that emits and receives radio waves receives reflected electromagnetic energy at the same frequency across a time interval (t).

Moreover, the radio frequency signals are constantly imparted over analyte molecules that relate to blood pressure with increasing frequency which causes the molecules to charge up or get polarized, and pressure ions of the molecules get displaced. The analytes that relate to blood pressure may be ions that require relaxation time to get back to their original state. However, due to the regular transmission of radio frequency signals, the analytes that relate to blood pressure may be ions that do not get enough time to return to their original state and remain charged. The receiving antenna receives distorted reflected radio frequency signals from these highly polar or charged analytes that relate to blood pressure. Therefore, there is a need for an improved system and method to train a model for monitoring health parameters with enhanced accuracy.

### SUMMARY

An improved health monitoring system and method for monitoring health parameters with enhanced accuracy. In an embodiment, the health monitoring system and method can be used to train a model. In an embodiment, a method for training a model to monitor the health parameters of a user includes monitoring a blood pressure of a user using a control blood pressure monitoring system; receiving control data corresponding to the monitoring using the control blood pressure monitoring system; inputting a frequency range and a frequency-time event and calculating a randomized frequency range; incrementing the frequency-time event; transmitting the calculated randomized frequency and matching the frequency-time limit with the stored frequency-time limit; receiving the calculated randomized frequency range corresponding to radio waves that have reflected from blood in a blood vessel of the user; generating training data by combining the control data with the randomized frequency range in a time synchronous manner; and training a model using the training data. The trained model correlates frequency range to values indicative of the user's blood pressure.

One example of a health monitoring system can include a monitoring device that includes one or more transmit antennas configured to transmit radio-frequency (RF) analyte detection signals into a user suitable for detecting an analyte in the user, and one or more receive antennas configured to detect reflected RF analyte signals that result from the RF analyte detection signals transmitted into the user. An analog-to-digital converter may be connected to the one or more receive antennas and receives the reflected RF analyte signals detected by the one or more receive antennas in order to convert the detected RF analyte signals from analog signals to digital signals. In addition, a randomized frequency generator may be connected to the one or more transmit antennas that generates the RF analyte detection signals having randomized frequencies. The random frequencies of the RF analyte detection signals allow more relaxation time to allow polar molecules excited by the RF analyte detection signals to return to their original state.

Another example of a health monitoring method can include detecting an analyte in a user by transmitting radio-frequency (RF) analyte detection signals having randomized frequencies into the user from one or more transmit antennas and detecting, using one or more receive antennas, RF analyte signals that result from the RF analyte detection signals transmitted into the user. The detected RF analyte signals are converted from analog signals to digital signals using an analog-to-digital converter connected to the one or more receive antennas.

A method for training a model to monitor the health parameter of a user can include monitoring a blood pressure of a user using a control blood pressure monitoring system; receiving control data corresponding to the monitoring using the control blood pressure monitoring system; inputting a frequency range and a frequency-time event and calculating a randomized frequency range; transmitting a calculated randomized frequency through at least one antenna and matching a frequency-time limit with a stored frequency-time limit; receiving calculated randomized frequency range data corresponding to radio waves that have reflected from blood of the user; generating training data by combining the control data with the randomized frequency range data in a time synchronous manner; and training a model using the training data to produce a trained model, wherein the trained model correlates randomized frequency range data to values indicative of the user's blood pressure.

### DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A illustrates a prior art frequency versus time graph of transmitted radio waves in a stepped frequency transmission approach;

FIG. 1B illustrates a prior art amplitude versus time graph of transmission waveforms;

FIG. 2 illustrates a frequency versus polarizability graph, according to an embodiment;

FIG. 3 illustrates a bar chart depicting a randomized frequency transmission approach, according to an embodiment;

FIG. 4 illustrates a block diagram of a control blood pressure monitoring system for training a model using the randomized frequency transmission approach, according to an embodiment;

FIG. 5 illustrates a block diagram of internal components of a device base module of the control blood pressure monitoring system, according to an embodiment;

FIG. 6 illustrates a flowchart of a method executed by an initializing module, according to an embodiment;

FIG. 7 illustrates a flowchart of a method executed by a randomized frequency module, according to an embodiment;

FIG. 8 illustrates a flowchart of a method executed by a set frequency module, according to an embodiment;

FIG. 9 illustrates a flowchart of a method executed by an execute module, according to an embodiment; and

FIG. 10 illustrates a flowchart of a method executed by a checksum module, according to an embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

US 2021/0030282 is incorporated herein by reference in its entirety.

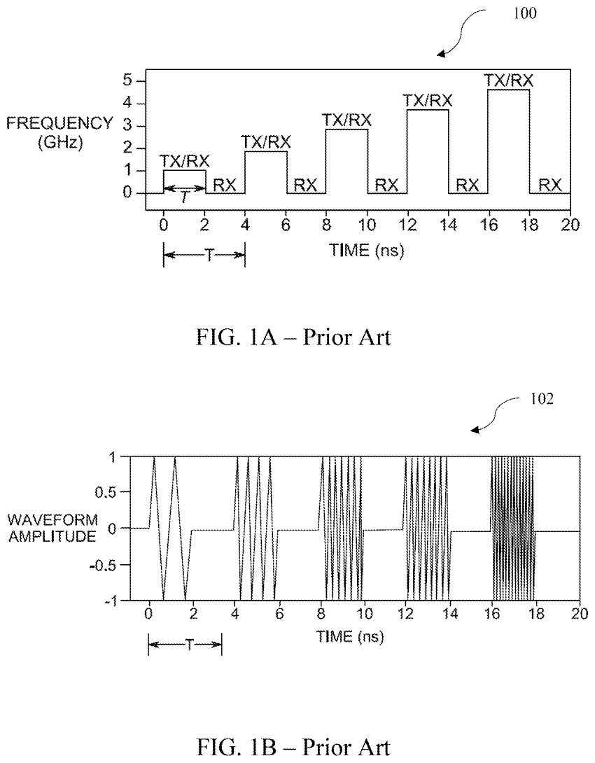

FIG. 1A illustrates a prior art frequency versus time graph 100 of transmitted radio waves in a stepped frequency transmission approach, according to an embodiment.

The transmitted radio waves correspond to transmission pulses transmitted in the stepped frequency transmission approach. The graph 100 depicts the frequency versus time of transmission pulses, with transmit (TX) intervals and receive (RX) intervals identified relative to the transmission pulses. The frequency at which the radio waves are transmitted through a user's skin is gradually increased, in the stepped frequency transmission approach, as shown in the graph 100. The increase in the frequency represented as Δf may be constant with every repetition interval (T). For example, for a first pulse, a transmitting antenna sends an RF frequency wave at 1 GHz between a time interval of 0-2 nanoseconds (ns). Further, a receiving antenna receives the reflected radio waves between 2-4 ns. Similarly, for the next pulse, the transmitting antenna sends another radio frequency wave at 2 GHz between a time interval of 4-6 ns, and the receiving antenna receives the reflected radio waves between 6-8 ns. This change in frequency keeps on increasing with every time interval.

FIG. 1B illustrates a prior art amplitude versus time graph 102 of transmission waveforms, according to an embodiment.

The amplitude of the transmission pulses is constant compared to the frequency that increases by Δf (delta f) at each repetitive time interval T. For example, in a first pulse, radio waves are transmitted at waveform amplitude in a range of −1 to 1 at a time interval of 0-4 ns. The frequency at the time interval of 0-4 ns may be (f). Further, in a second pulse, radio waves are transmitted at waveform amplitude in a range of −1 to 1 at a time interval of 4-8 ns. The frequency at the time interval of 4-8 ns may be (f+Δf). The increase in the frequency with the change in the time interval is considered as beaming of the radio waves, which may charge up polar blood pressure related analytes or molecules. It can be noted that the polar blood pressure related analytes or molecules act as dielectric when impacted by the radio frequency signals. The charging of the blood pressure related analytes that are polar molecules thereby acts as a drawback in determining the user's blood pressure level.

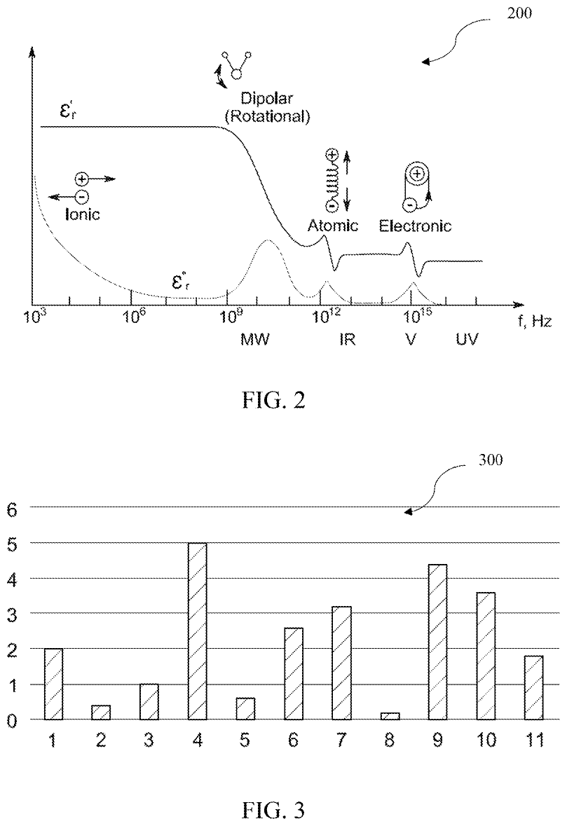

FIG. 2 illustrates a frequency versus polarizability graph 200, according to an embodiment.

The frequency versus polarizability graph 200 describes polarizations of dielectric materials such as blood pressure related molecules. The dielectric molecules exhibit three polarizations in different frequency regions. The three polarizations include electronic polarization, ionic polarization, and orientational or atomic polarization. The different frequency regions include a microwave (MW) region, an infrared (IR) region, a violet (V) region, and an ultraviolet (UV) region. The frequency versus polarizability graph 200 depicts the different frequency regions along the ordinate and the three polarizations along the abscissa. The MW region exhibits a frequency range from 1 kHz to 1 GHz, the IR region exhibits a frequency range from 1 GHz to 1000 GHz, and the UV region exhibits a frequency range from 1000 GHz to 1000000 GHz. The ionic polarization exists in the MW region, the orientational polarization from the MW region to the IR region, and the electronic polarization from the IR region to the UV region.

In the ionic polarization, ions get displaced; therefore, more relaxation time is required to allow them to return to their original state. Typically, in the step frequency transmission approach, the radio frequency signals are constantly imparted over blood pressure related analytes or “blood pressure related molecules” with increasing frequency which cause the molecules to charge up or get polarized, and blood pressure related ions or molecules get displaced. The blood pressure related ions or molecules require relaxation time to get back to their original state. Also, due to the regular transmission of radio frequency signals, the blood pressure related ions do not get enough time to return to their original state and remain charged. The receiving antenna receives a distorted reflected radio frequency signals from these highly polar or charged blood pressure related ions or molecules.

Further, the blood pressure related ions or molecules exhibit dielectric properties when the radio frequency signals are transmitted. The transmission of the radio waves on various frequencies causes a charging effect over the blood pressure related ions or molecules. As discussed earlier, the stepped frequency transmission approach involves transmitting radio waves by stepping up the frequencies. This causes charging up of the blood pressure related ions or molecules. It can be noted that blood pressure related ions or molecules are highly polar molecules with hydroxyl (OH) groups all over them, which are themselves very polar, as an oxygen atom is more electronegative than hydrogen. Therefore, the blood pressure related ions or molecules shifts electron density in the OH bond, making the Hydrogen delta + and O delta −. It can also be noted that the blood pressure related ions or molecules are also very unsymmetrical, which tends to make them polar. Therefore, a receiving antenna in the step frequency transmission approach may receive distorted reflected radio frequency signals with the highly polar blood pressure related ions or molecules and thereby provides distorted or wrong measurements of the blood pressure level.

FIG. 3 illustrates a bar chart 300 depicting a randomized frequency transmission approach, according to an embodiment.

The randomized frequency transmission approach is different compared to the stepped frequency transmission approach. The randomized frequency transmission approach involves transmitting the radio frequency signals over the blood pressure related molecules using randomized frequencies. For example, in a first pulse, a radio wave is transmitted by a transmitting antenna (not shown) over a target vein (not shown) of a user at a random frequency of 2 GHz in a time frame of 0-1 ns. In a second pulse, another radio wave is transmitted over the target vein of the user at the random frequency of 0.3 GHz in the time frame of 1-2 ns. In a third pulse, another radio wave is transmitted over the target vein of the user at the random frequency of 1 GHz in the time frame of 2-3 ns.

In one embodiment, the transmitting antenna may be configured to transmit the radio frequency signal in a first instant at 5 GHz and a second instant at 1 GHz. This sudden drop in the frequency of the radio frequency signals provides relaxation time for the ions of the blood pressure related ions or molecules to reach their original state after displacement due to a polarization effect. It can be noted that the randomized frequency transmission approach does not charge up the blood pressure related ions or molecules for a given time range. Therefore, the randomized frequency transmission approach provides an advantage over the stepped frequency transmission approach.

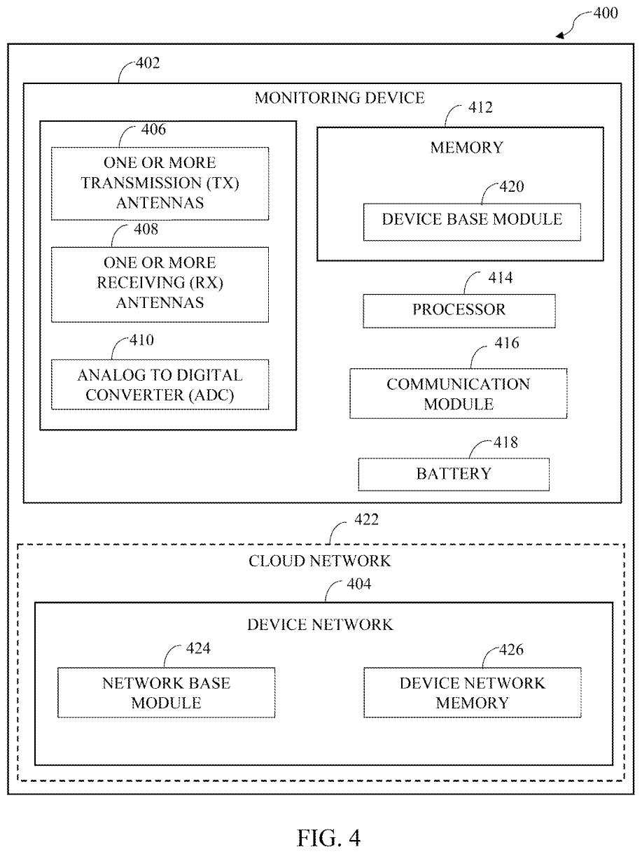

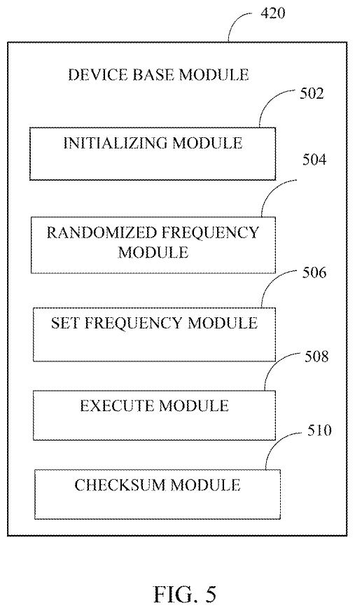

FIG. 4 illustrates a block diagram of a control blood pressure monitoring system 400 for training a model using the randomized frequency transmission approach, according to an embodiment. FIG. 4 is described in conjunction with FIGS. 5-10. The control blood pressure monitoring system 400 is referred to as a system 400.

The system 400 may comprise a monitoring device 402 communicatively coupled to a device network 404. In one embodiment, the device network 404 may be a wireless and/or wired communication channel. The monitoring device 402 may be worn by the user. The monitoring device 402 may be configured to determine health parameters using the radio frequency signals and based upon which generating control data that corresponds to the monitoring of the blood pressure level. Control data is obtained from a blood pressure cuff or other standard means and stored in memory so that the monitoring device 402 can relate the control data to the data collected using its received antenna data. In this way, control data from a standard source can be correlated to the responded radio frequencies from the user. The system 400 may target specific blood vessels using the radio frequency signals, and the responded or output signals may correspond to the blood pressure level in the user.

In one embodiment, the system 400 may include integrated circuit (IC) devices (not shown) with transmit and/or receive antennas integrated therewith. Monitoring the blood pressure level in the specific blood vessels of the user using the radio frequency signals involves the transmission of suitable radio frequency signals below the user's skin surface. Corresponding to the transmission, a reflected portion of the radio frequency signals is received on multiple receive antennas. Further, the system 400 isolates and/or processes a signal from a particular location of the blood vessels in response to the received radio frequency signals. The system 400 may output a signal from the received radio frequency signals that correspond to the blood pressure level in the user. It can be noted that the monitoring device 402 may be worn by the user at various locations such as wrist, arm, leg, etc.

In one embodiment, the system 400 for monitoring the blood pressure level of the user using the radio frequency signals involves transmitting radio frequency signals below the skin surface, receiving a reflected portion of the radio frequency signals on multiple receive antennas, isolating a signal from the radio frequency signals at a particular location in response to the received radio frequency signals, and outputting a signal that corresponds to the blood pressure level in the user in response to the isolated signal. In one embodiment, beamforming is used in the receiving process to isolate the radio frequency signals responded from the analyte from the blood of a specific location on a specific blood vessel to provide a high-quality signal corresponding to the blood pressure levels in the specific blood vessel. In another embodiment, Doppler effect processing may be used to isolate the radio frequency signals reflected from the the analyte from the blood of specific blood vessel's specific location to provide the high-quality signal corresponding to the blood pressure levels in the specific blood vessel. It can be noted that analog and/or digital signal processing techniques may be used to implement beamforming and/or Doppler effect processing and digital signal processing of the received signals. In another embodiment, the beamforming and the Doppler effect processing may be used together to isolate the radio frequency signals responded from the analyte from the blood of specific location in the specific blood vessel to provide the high-quality signal corresponding to the blood pressure levels in the specific blood vessel.

In one exemplary embodiment, radio frequency signals of a higher frequency range of 122-126 gigahertz (GHz) having a shallower penetration depth are used to monitor blood pressure levels. It can be noted that the shallower penetration depth reduces undesirable reflections, such as reflections from bone, and dense tissue, such as tendons, ligaments, and muscle, which may reduce the signal processing burden and improve the quality of the desired signal generated from the location of the blood vessel. It can also be noted that bones are dielectric and semi-conductive. In addition, bones are anisotropic, so not only are bones conductive and conduct differently depending on the direction of the flow of current through the bone. Alternatively, the bones are also piezoelectric materials. Therefore, radio frequency signals of higher frequency range of 122-126 GHz with the shallower penetration depth are used to monitor the blood pressure levels.

Further, the monitoring device 402 may comprise one or more transmission (TX) antennas 406, one or more receiving (RX) antennas 408, an analog to digital converter (ADC) 410, a memory 412, a processor 414, a communication module 416 and a battery 418. In one embodiment, the monitoring device 402 may be a wearable and portable device such as, but not limited to, a cell phone, a smartwatch, a tracker, a wearable monitor, a wristband, and a personal blood monitoring device. The one or more TX antennas 406 and the one or more RX antennas 408 may be fabricated on a substrate (not shown) within the monitoring device 402 in a suitable configuration. In one exemplary embodiment, at least two TX antennas and at least four RX antennas are fabricated on the substrate. The one or more TX antennas 406 and the one or more RX antennas 408 may correspond to a circuitry arrangement (not shown) on the substrate. Further, the ADC 410, the memory 412, the processor 414, the communication module 416, and the battery 418 may be fabricated on the substrate. Further, the communication module 416 may be configured to facilitate communication between the monitoring device 402 and the device network 404.

Further, the one or more TX antennas 406 and the one or more RX antennas 408 may be integrated into the circuitry arrangement. The one or more TX antennas 406 may be configured to transmit the radio frequency signals at a predefined frequency. In one embodiment, the predefined frequency may correspond to a range suitable for the human body. For example, the one or more TX antennas 406 transmit radio frequency signals at 122-126 GHz. Successively, the one or more RX antennas 408 may be configured to receive the reflected portion of the radio frequency signals.

In one embodiment, the radio frequency signals may be transmitted into the user, and electromagnetic energy may be reflected from many parts such as fibrous tissue, muscle, tendons, bones, and the skin. It can be noted that effective monitoring of the blood pressure level is facilitated by an electrical response of blood pressure related molecules, such as pancreatic endocrine hormones, against the transmitted radio frequency signals. It will be apparent to a skilled person that the pancreatic endocrine hormones such as insulin and glucagon are responsible for maintaining sugar or pressure level. Further, the electromagnetic energy reflected from the blood pressure related molecules may be received by the one or more RX antennas 408. Further, the ADC 410 may be coupled to the one or more RX antennas 408. The one or more RX antennas 408 may be configured to receive the reflected radio frequency signals. The ADC 410 may be configured to convert the received radio frequency signals from an analog signal into a digital processor readable format.

Further, the memory 412 may be configured to store the transmitted radio frequency signals by the one or more TX antennas 406 and receive a reflected portion of the transmitted radio frequency signals from the one or more RX antennas 408. Further, the memory 412 may also store the converted digital processor readable format by the ADC 410. In one embodiment, the memory 412 may include suitable logic, circuitry, and/or interfaces that may be configured to store a machine code and/or a computer program with at least one code section executable by the processor 414. Examples of implementation of the memory 412 may include, but are not limited to, Random Access Memory (RAM), Read Only Memory (ROM), Hard Disk Drive (HDD), and/or a Secure Digital (SD) card.

Further, the system 400 may comprise a device base module 420 fabricated within the memory 412. The device base module 420 may be configured to initiate the processor 414 to generate randomized frequency scanning data. The randomized frequency scanning data may be generated from the signals reflected from the user's target vein when the radio frequency signals are transmitted at randomized frequencies. Further, the randomized frequency scanning data may be used to generate training data that is further used to produce a trained model. The internal components and detailed working of the device base module 420 are described later in conjunction with FIG. 5. Further, the device base module 420 may be configured to store a set of instructions for executing the computer program from the converted digital processor readable format of the ADC 410. The device base module 420 is configured to facilitate the operation of the processor 414, the memory 412, the one or more TX antennas 406, the one or more RX antennas 408, and the communication module 416.

Further, the processor 414 may facilitate operation of the monitoring device 402 with the device network 404 to perform functions according to the instructions stored in the memory 412. In one embodiment, the processor 414 may include suitable logic, circuitry, interfaces, and/or code that may be configured to execute a set of instructions stored in the memory 412. The processor 414 may be configured to run the instructions obtained by the device base module 420 to generate the randomized frequency scanning data.

In one embodiment, the processor 414 may be configured to produce control data that corresponds to monitoring the user's blood pressure level by using the monitoring device 402. In one embodiment, the control data corresponds to the blood pressure monitored by the user by using a conventional range of radio waves that are penetrated into the user's skin. In one embodiment, the system 400 may further be configured to generate the training data by combining the randomized frequency scanning data and the control data saved in the memory 412. The processor 414 may collect real-time signals from the one or more TX antennas 406, and the one or more RX antennas 408 and may store the real-time signals in the memory 412 and, based upon which, generate the control data. In one embodiment, the real-time signals may be assigned as initial and updated radio frequency (RF) signals. Examples of the processor 414 may be an X86-based processor, a Reduced Instruction Set Computing (RISC) processor, an Application-Specific Integrated Circuit (ASIC) processor, a Complex Instruction Set Computing (CISC) processor, and/or other processors. The processor 414 may be a multicore microcontroller designed to carry multiple operations based on predefined algorithm patterns to achieve a desired result.

Further, the processor 414 may take inputs from the monitoring device 402 and retain control by sending signals to different parts of the monitoring device 402. The processor 414 may include a Random Access Memory (RAM) that is used to store data and other results created when the processor 414 is at work. It can be noted that the data is stored temporarily for further processing, such as filtering, correlation, correction, and adjustment, and the generated control data and randomized frequency scanning data are stored permanently in the memory 412. Moreover, the processor 414 carries out special tasks as programs that are pre-stored in the Read Only Memory (ROM). It can be noted that the special tasks carried out by the processor 414 indicate and apply certain actions which trigger specific responses.

Further, the communication module 416 of the monitoring device 402 may communicate with the device network 404 via a cloud network 422. Examples of the communication module 416 may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). In one embodiment, various devices may be configured to have a communication module integrated over circuitry arrangement to connect with the device network 404 via various wired and wireless communication protocols, such as the cloud network 422. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols. In one embodiment, the battery 418 may be disposed over the substrate to power hardware modules of the monitoring device 402. The monitoring device 402 may be configured with a charging port to recharge the battery 418. It can be noted that the charging of the battery 418 may be achieved using wired or wireless means. In one embodiment, the battery 418 may include different models of lithium-ion batteries, such as CR1216, CR2016, CR2032, CR2025, CR2430, CR1220, CR1620, and CR1616.

Further, the system 400 may be configured to provide communication for transmitting and receiving signals between the monitoring device 402 and the device network 404. The device network 404 may comprise a network base module 424 and a device network memory 426.

The device network 404 may be configured to receive the control data and the randomized frequency scanning data from the device base module 420 using the communication module 416. The control data and the randomized frequency scanning data of the device base module 420 may be transmitted to the network base module 424. Examples of the network base module 424 may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). Further, the device network 404 comprises the device network memory 426 configured to store the control data and the randomized frequency scanning data received from the monitoring device 402. Examples of implementation of the device network memory 426 may include, but are not limited to, Cloud storage, Cloud server, Random Access Memory (RAM), Read Only Memory (ROM), and/or a Secure Digital (SD) card.

FIG. 5 illustrates a block diagram of the internal components of the device base module 420, according to an embodiment.

The device base module 420 may work synchronously with the processor 414 to generate the randomized frequency scanning data by transmitting radio frequency signals over the target vein of the user at randomized frequencies. The device base module 420 may comprise an initializing module 502, a randomized frequency module 504, a set frequency module 506, an execute module 508, and a checksum module 510. In one embodiment, the initializing module 502, the randomized frequency module 504, the set frequency module 506, the execute module 508, and the checksum module 510 may execute sequentially to generate the randomized frequency scanning data.

In one embodiment, the initializing module 502 may be configured to allow input of a frequency range and frequency-time event in the memory 412. The frequency range may be described as the lowest and highest limit of the frequency at which the one or more TX antennas 406 transmit the radio frequency signals over the target vein of the user. For example, the one or more TX antennas 406 may be configured to transmit the radio frequency signals between standard frequency of 1-200 GHz frequency over the target vein in a randomized manner.

Further, the frequency-time event may be a time frame for which the randomized radio frequency signals are to be transmitted over the target vein of the user. For example, one or more TX antennas 406 may be configured to transmit radio frequency signals at a random frequency from 0-200 GHz between 0-2 ns. Further the one or more TX antennas 406 may be configured to transmit another radio frequency signal at the randomized frequency from the frequency range of 1-200 GHz between 2-4 ns. It may be noted that the initializing module 502 may clear the previously saved frequency range and frequency-time event from the memory 412, if any, before enabling input of a new frequency range and new frequency-time event. The previously saved frequency range may be referred to as a pre-saved frequency range. The pre-saved frequency range may be updated for each frequency-time event.

In one embodiment, the randomized frequency module 504 may be configured to calculate the randomized frequencies from the pre-saved frequency range. The randomized frequency module 504 may randomly calculate a single frequency from the pre-saved frequency range at which the radio frequency signal is to be transmitted by the one or more TX antennas 406. For example, the randomized frequency module 504 calculates 5 GHz frequency for the one or more TX antennas 406 to transmit over the target vein of the user.

Further, the randomized frequency module 504 may be configured to determine if the calculated frequency has already been used by the monitoring device 402 in the current frequency range. The frequency calculated by the randomized frequency module 504 is compared with the pre-saved frequencies in the memory 412 that have been previously used. In one case, if the calculated frequency does not match the pre-saved frequencies in the memory 412, the randomized frequency module 504 may be configured to store the calculated frequency in the memory 412. In another case, if the calculated frequency matches one of the pre-saved frequencies in memory 412, the randomized frequency module 504 may be configured to re-calculate the random frequency.

In one example, the randomized frequency module 504 calculates a random frequency of 5 GHz and compares it with pre-saved random frequencies of 2 GHz, 3.4 GHz, 80 GHZ, and 27 GHz. The calculated random frequency of 5 GHz does not match any pre-saved randomized frequencies. Therefore, the randomized frequency module 504 initiates to store the 5 GHz frequency with the memory 412. In another example, the randomized frequency module 504 calculates a randomized frequency of 80 GHz and matches with the pre-saved randomized frequencies of 2 GHz, 3.4 GHz, 80 GHz, and 27 GHz. Therefore, the randomized frequency module 504 re-calculates another randomized frequency.

Further, the set frequency module 506 may be configured to increment the frequency-time event and store the frequency-time event. The set frequency module 506 may be configured to increment the frequency-time event inputted by the initializing module 502. The set frequency module 506 may set a frequency-time range for the frequency-time event. The frequency-time range may be described as the total time interval for which the radio frequency signals at randomized frequencies are transmitted by the one or more TX antennas 406. For example, the set frequency module 506 increments a 30-second frequency-time range with a frequency-time event of 2 ns. In one embodiment, the set frequency module 506 may be configured to store the frequency-time range and the frequency-time event in the memory 412.

Further, the execute module 508 may be configured to send the radio frequency signals at the stored randomized frequencies for the stored frequency-time range and the frequency-time event. For example, the execute module 508 may transmit a first pulse of radio frequency signal at 5 GHz for 0-2 ns and a second pulse at 2 GHz for 2-4 ns. In one embodiment, the execute module 508 may be configured to measure the reflected radio frequency signals from the target vein. For example, the execute module 508 receives a reflected radio frequency signal of 4 GHz in response to the transmitted 5 GHz radio frequency signal. Further, the execute module 508 may be configured to store the received radio frequency signal along with the randomized frequency, the frequency range, and the frequency-time event. For example, the execute module 508 stores a received radio frequency signal at 4 GHz, the randomized frequency of 5 GHz, the frequency range of 0-30 seconds, and the frequency-time event of 2 ns.

Further, the checksum module 510 may be configured to determine whether the stored frequency-time range is met. For example, the checksum module 510 determines that the radio frequency signal of the randomized frequencies has been transmitted for 30 seconds. In one case, if the randomized frequencies are transmitted for 30 seconds, the operation of the randomized frequency module 504 is terminated. In another case, if the randomized frequencies are transmitted for less than 30 seconds, the randomized frequency module 504 initiates generating another randomized frequency.

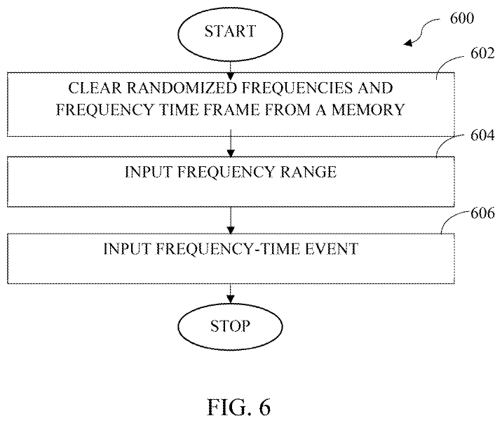

FIG. 6 illustrates a flowchart of a method 600 executed by the initializing module 502, according to an embodiment.

At first, the initializing module 502 may be configured to clear the randomized frequencies and the frequency-time frame from the memory 412 at step 602. In an embodiment, the initializing module 502 may clear the previously saved frequency range and frequency-time event from the memory 412, if any, before enabling input of the new frequency range and frequency-time event. For example, the initializing module 502 clears the randomized frequencies of 2 GHz, 6 GHZ, 45 GHz, 30 GHz, and 5 GHz, and the frequency-time frame of 5 ns from the memory 412.

Successively, the initializing module 502 may be configured to receive the input frequency range, from memory 412, at step 604 and input frequency-time event, at step 606. For example, the initializing module 502 may be configured to input the input frequency range of 1-200 GHz and the frequency-time event of 0-2 ns in the 0-30 seconds time frame over the target vein in a randomized manner, using one or more TX antennas 406.

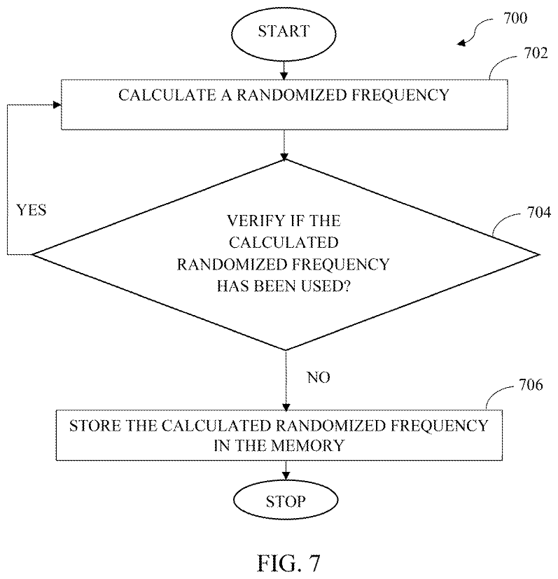

FIG. 7 illustrates a flowchart of a method 700 executed by the randomized frequency module 504, according to an embodiment.

At first, the randomized frequency module 504 may be configured to calculate a random frequency at step 702. For example, the randomized frequency module 504 calculates 5 GHz frequency between the input frequency range of 1-200 GHz at which the one or more TX antennas 406 transmits the radio frequency signal over the target vein of the user.

Successively, the randomized frequency module 504 may be configured to verify if the calculated randomized frequency has been used at step 704. The randomized frequency module 504 may be configured to determine if the calculated randomized frequency is already used by the monitoring device 402. In one case, the randomized frequency module 504 may verify that the calculated randomized frequency has been used. In this case, the randomized frequency module 504 is redirected back to step 702, to re-calculate another randomized frequency. For example, the randomized frequency module 504 verifies that the calculated randomized frequency of 5 GHz is already present within the pre-saved frequencies of 2 GHz, 3.4 GHz, 5 GHz, 80 GHz, and 27 GHz.

In another case, the randomized frequency module 504 may verify that the calculated randomized frequency has not been used. In this case, the randomized frequency module 504 proceeds to step 706 to store the calculated randomized frequency in the memory 412. For example, the randomized frequency module 504 verifies that the calculated randomized frequency of 5 GHz is not present within the pre-saved frequencies of 2 GHz, 3.4 GHz, 80 GHZ, and 27 GHz. In one embodiment, the process of picking a random frequency continues, until all frequencies are used in the range. Each time a random frequency is created, its stored and later each new random frequency generated is checked against what is stored.

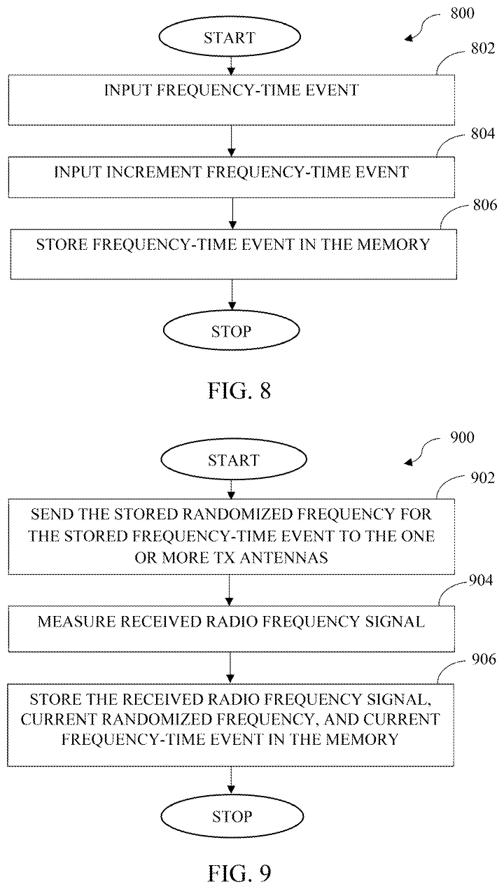

FIG. 8 illustrates a flowchart of a method 800 executed by the set frequency module 506, according to an embodiment.

At first, the set frequency module 506 may be configured to input the frequency-time event and increment frequency-time event at step 802 and step 804, respectively. For example, the set frequency module 506 inputs 0-30 seconds as the frequency-time range (step 802) in which the frequency-time event of 2 ns is incremented (step 804). Successively, the set frequency module 506 may be configured to store the frequency-time event in the memory 412 at step 806. For example, the set frequency module 506 stores the frequency-time event of 2 ns seconds and the frequency-time range of 0-30 seconds in the memory 412.

FIG. 9 illustrates a flowchart of a method 900 executed by the execute module 508, according to an embodiment.

At first, the execute module 508 may be configured to send the stored randomized frequency for the stored frequency-time event to the one or more TX antennas 406 at step 902. For example, the execute module 508 sends the stored randomized frequency of 5 GHz for a stored frequency-time event of 0-2 ns to the one or more TX antennas. Successively, the execute module 508 may be configured to measure the received radio frequency signal at step 904. For example, the execute module 508 measures that the received reflected radio frequency signal is 4 GHz in response to the transmitted 5 GHz radio frequency signal.

Successively, the execute module 508 may be configured to store the received radio frequency signal, current randomized frequency, and current frequency-time event in the memory 412, at step 906. For example, execute module 508 stores the received radio frequency signal of 4 GHz, the current randomized frequency of 5 GHz, and the current frequency-time event of 2 ns.

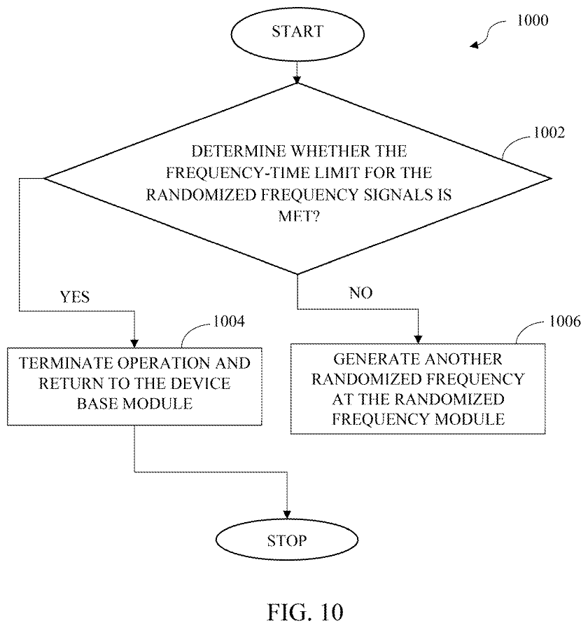

FIG. 10 illustrates a flowchart of a method 1000 executed by the checksum module 510, according to an embodiment.

At first, the checksum module 510 may be configured to determine whether the frequency-time limit for the randomized frequency signals is met at step 1002. In one case, the checksum module 510 may determine that the frequency-time limit for the randomized frequency signals is met. In this case, the checksum module 510 may proceed to step 1004, to terminate operation and return to the device base module 420. For example, the checksum module 510 determines that the radio frequency signal with randomized frequencies from 1-200 GHz has been transmitted for 30 seconds.

In another case, the checksum module 510 may determine that the frequency-time limit for the randomized frequency signals is not met. In this case, the checksum module 510 may proceed to step 1006 to generate another randomized frequency at the randomized frequency module 504. For example, the checksum module 510 determines that the radio frequency signal with randomized frequencies from 1-200 GHz has been transmitted for 26 seconds which is less than the frequency-time limit of 30 seconds.

It will be appreciated by those skilled in the art that changes could be made to the exemplary embodiments described above without departing from the broad inventive concept thereof. It is to be understood, therefore, that this disclosure is not limited to the particular embodiments disclosed, but is intended to cover modifications within the spirit and scope of the subject disclosure as disclosed above.

1. A health monitoring system, comprising:
a monitoring device that includes one or more transmit antennas configured to transmit radio-frequency (RF) analyte detection signals into a user suitable for detecting an analyte in the user, and one or more receive antennas configured to detect reflected RF analyte signals that result from the RF analyte detection signals transmitted into the user;
an analog-to-digital converter connected to the one or more receive antennas and receiving the reflected RF analyte signals detected by the one or more receive antennas; and
a randomized frequency generator connected to the one or more transmit antennas that generates the RF analyte detection signals having randomized frequencies.

2. The health monitoring system of claim 1, wherein the analog-to-digital converter and the randomized frequency generator are part of the monitoring device.

3. The health monitoring system of claim 1, wherein the analyte comprises a blood pressure related analyte.

4. The health monitoring system of claim 3, wherein the blood pressure related analyte comprises a polar molecule.

5. The health monitoring system of claim 1, wherein the randomized frequency generator is configured to generate the RF analyte detection signals having randomized frequencies based on an input frequency range and an input frequency-time event.

6. The health monitoring system of claim 1, further comprising a control monitoring system that generates control data.

7. The health monitoring system of claim 6, further comprising a training data generator connected to the control monitoring system and to the monitoring device.

8. A health monitoring method, comprising:
detecting an analyte in a user by transmitting radio-frequency (RF) analyte detection signals having randomized frequencies into the user from one or more transmit antennas and detecting, using one or more receive antennas, RF analyte signals that result from the RF analyte detection signals transmitted into the user;
converting the detected RF analyte signals from analog signals to digital signals using an analog-to-digital converter connected to the one or more receive antennas.

9. The health monitoring method of claim 8, wherein the analyte comprises a blood pressure related analyte.

10. The health monitoring method of claim 9, wherein the blood pressure related analyte comprises a polar molecule.

11. The health monitoring method of claim 8, comprising generating the RF analyte detection signals having randomized frequencies using a randomized frequency generator that generates the RF analyte detection signals having randomized frequencies based on an input frequency range and an input frequency-time event.

12. The health monitoring method of claim 8, further comprising generating control data using a control monitoring system.

13. The health monitoring method of claim 12, further comprising generating training data by combining the control data with the digital signals in a time synchronous manner.

14. The health monitoring method of claim 13, further comprising training a model using the training data to produce a trained model.
